(19) United States
(12) Patent Application Publication
(10) Pub. No.: US 2008/0255665 A1
(43) Pub. Date: Oct. 16, 2008

(54) ANCHORED PROSTHETIC MENISCUS DEVICE

(75) Inventor: Noam Weissberg, Netanya (IL)

Correspondence Address: HAYNES AND BOONE, LLP, 901 Main Street, Suite 3100, Dallas, TX, 75202, US

(73) Assignee: Active Implants Corporation, Memphis, TN (US)

(21) Appl. No.: 11/734,052

(22) Filed: Apr. 11, 2007

(51) Int. Cl. A61F 2/30; A61F 2/38; A61F 2/08

(52) U.S. Cl. 623/14.12; 623/908

(57) ABSTRACT

A human implantable meniscus device having a unique anchoring system for locking the device into a bone and reinforcing fibers in order to mimic the mechanical characteristics of natural meniscus.

### BACKGROUND OF THE INVENTION

1. Field of the Invention

The disclosure generally relates to a human implantable prosthetic meniscus device having a unique anchoring system for locking the device into a bone and reinforcing fibers in order to mimic the mechanical characteristics of natural meniscus,

2. Description of Related Art

The human knee joint contains a medial meniscus, and a lateral meniscus. The lateral meniscus is located on the outer side of the leg, directly above the location where the fibula condoyle is coupled to the tibia. The medial meniscus is located on the inner side of the leg.

Each meniscus is a crescent-shaped fibrocartilaginous tissue primarily attached to the tibial bone at an anterior and a posterior horn. Each meniscus has a wedged shaped cross section and a substantially larger curvature and arc. The thickest region is around the periphery also known as the circumference or the rim.

The inner edge of a meniscus is the thinnest portion of the wedge; this edge is also known as the apex or the margin. The apex is not anchored the tibia, instead, as the person walks or runs, each meniscus in the knee is somewhat free to move, as it is squeezed between the tibial plateau (beneath it) and a femoral runner (above it). The bottom surface of each meniscus is relatively flat, so it can ride in a relatively stable manner on top of the tibial plateau. The top surface is concave, so it can provide better, more closely conforming support to the rounded edge of the femoral condoyle. Due to its shape, location, and ability to flex and move somewhat as it is pushed, each meniscus helps support and stabilize the outer edge of the femoral condoyle as it presses, slides and articulates against the portion of the tibial plateau beneath it.

The human knees are high-stress locations, where the medial and lateral menisci together act as a crucial stabilizer, a mechanism for force distribution, and a lubricant in the area of contact between the tibia and femur. The meniscii are subjected to frequently-repeated combinations of compression and tension. As a result one or both meniscii are often damaged, particularly in active people, requiring surgery to repair the damaged area.

Various efforts have been made, using prior technology, to repair or replace damaged meniscal tissue. However, because of the complex structures and anchoring involved, and because of the need to create and sustain extremely smooth and constantly wet surfaces on the inner portions of each meniscal wedge, prior methods of replacing or repairing damaged meniscal are not entirely adequate.

Treatment of injured or diseased menisci has generally been both by surgical repair and by excision. With excision, regeneration of meniscal tissue may occur. It is known that meniscal fibrochondrocytes have the ability to migrate into a defect filled with a fibrin clot and form tissue apparently similar to normal meniscal fibrocartilage. When an adequate matrix scaffold is present within a meniscal defect, such meniscal fibrocartilage may be formed. Meniscal tissue is also capable of self-repair when exposed to bleeding tissues, and additionally, it is also known in the prior art that meniscal cells in tissue culture are capable of cell division and matrix synthesis. Replacement of an injured meniscus in an otherwise healthy joint may prevent arthritic changes and may stabilize the joint.

In diseased joints, replacement of the meniscus may reduce the progression of the disease process, and may provide pain relief. Allografting or meniscal transplantation, is one method of replacement which has been executed both in dogs and in humans. However, this approach has been only partially successful over the long term due to the host's immunologic response to the graft, to failures in the cryopreservation process, and to failures of the attachment sites.

Often though the meniscus is damaged in the apex area requiring removal of portions of the meniscus. In some cases the damage to the meniscus is beyond repair and complete removal of the meniscus is required. Damage alone to the meniscus can cause debilitating pain and arthritis. Complete removal of the meniscus can cause long term pain and eventually arthritis in the joint.

In alternative prior art replacement approaches, menisci have been replaced with prostheses composed of permanent artificial materials. Such prostheses have been constructed of purely artificial materials in order to minimize the possibility of an immunological response. In addition, the use of such materials is believed to be advantageous because it permits construction of a structure which can withstand the high and repeated loads which are encountered in the knee joint, and because it can alter the joint mechanics in beneficial ways that biological materials would not tolerate.

For example, a Teflon net has been used to replace the resected meniscus of a dog upon which fibrous ingrowth or regeneration was observed, although accompanied by significant chondral abrasion. A prosthetic meniscus has also been constructed from resilient materials such as silicone rubber or Teflon with reinforcing materials of stainless steel or nylon strands (U.S. Pat. No. 4,502,161). A meniscal component has also been made from resilient plastic materials (U.S. Pat. No. 4,085,466).

However, the replacement of meniscal tissue with structures consisting of permanent artificial materials generally has been unsuccessful, principally because the opposing articular cartilage of human and animal joints is fragile. The articular cartilage in the knee will not withstand abrasive interfaces, nor compliance variances from normal, which eventually results from the implantation of prior art artificial menisci. Additionally, joint forces are multiples of body weight which, in the case of the knee, are typically encountered over a million cycles per year. Thus far, prior art permanent artificial menisci have not been composed of materials having natural meniscal properties, nor have they been able to be positioned securely enough to withstand such routine forces.

For example U.S. Pat. No. 4,344,193, entitled Meniscus Prosthesis, teaches that the prosthetic device may be manufactured in varying sizes conforming to the varying sizes of the human body and, in addition, the inherent varying size of the inner and outer condoyle of the femur. However, the device disclosed is of the “floating” type which does not include an anchoring system to secure the prosthetic in place.

Both U.S. Pat. No. 5,092,894, Stabilized Meniscus Prosthesis, and U.S. Pat. No. 5,171,322, Stabilized Meniscal Prosthesis, teach a meniscus prosthetic device which includes a body and a tail which extends as a continuation of the body. The purpose of the tail, described therein, is to anchor the meniscus by threading the tail through a sleeve placed into predrilled bore holes in the tibial plateau. The sleeve allows for free movement of the tail ends with respect to the tibia. The tails are then secured to one another at the distal end of the bore holes to create a continues loop in numerous ways, including stitching and clips. Other means of securing the tails to the tibia are attempted including drilling a trough in the tibial plateau where the cross section of the trough is greater than the opening to the trough such that the tail ends may be forced into place in the trough. It is also disclosed to place the trough on the rim of the tibia.

Despite all the efforts cited above and numerous others in the field, as well, surgically placed prosthetic meniscii for damaged or diseased tissue suffer from a number of important limitations, including (1) relatively low strength and durability, and (2) difficulties in anchoring them permanently in a desired location, in ways that provide adequate strength and provide a relatively pain-free lifestyle for the recipient of the prosthetic. Both of these crucial factors severely limit the number and variety of uses for such prosthetic meniscus that have been developed and commercialized to date.

### SUMMARY OF THE INVENTION

Embodiments herein include a prosthetic meniscus device having an artificial meniscus body integrated with elongated artificial ligaments that mimic natural meniscus. The artificial ligaments include an anchoring system that locks the implant ligaments straight to the bone. Each integral ligament can be arranged to meet desired tensile values by being be drawn past an anchoring point within a surgically drilled bore having a diameter less than that of the anchoring system on the ligaments at least on a portion thereof.

The outer surface of the meniscus body can be made from lubricious material to maintain a lubricated feel in the knee joint. The prosthetic device may also include reinforced fibers to provide stabilization to the knee joint consistent with the mechanical characters of natural meniscus.

Since medial and lateral meniscus have different shapes and sizes, the prosthetic meniscus device according to the present invention can be sized and shaped in a customized manner to fit all patients.

Embodiments include a prosthetic meniscus device for implantation into a human knee between a plateau of a tibia having a pair of surgically drilled anchor holes and a femoral condoyle, the prosthetic meniscus device including a substantially semi-circular body portion having a substantially wedge shaped cross section positioned on the plateau of the tibia in contact with the femoral condoyle, the body portion have a first end and a second end, an elongated first and second anchor leads integrated with the body portion at the first and second ends respectively, each anchor lead having at least one anchor protuberance, where the anchor protuberance is structured and arranged to anchor the prosthetic meniscus to the tibia when the first and second anchor leads are respectively thread through corresponding first and second anchor holes in the tibia.

Embodiments further comprise anchor protuberances which include a flat cap surface positioned substantially perpendicular to the body portion and a conical surface sloping away from the body portion toward the distal end of the anchor lead.

In some embodiments each elongated anchor lead includes a plurality of anchor protuberances selectively spaced in order to achieve one of a set pre-determined tensile values in the anchor leads upon threading through the anchor holes in the tibia.

In some embodiments the body portion includes an outer surface made from a lubricious material and reinforcing fibers structured and arranged within the body portion and the elongated anchor leads.

Additional embodiments include anchor leads having a series threads structured and arranged to frictionally engage the walls of the bore holes drilled into the tibis.

Embodiments herein further include a prosthetic meniscus implant manufactured from resilient polymeric material comprising a generally semi-circular toroidal body having a first end and a second end and having a generally wedge shaped cross section whose apex generates an inferior generally arcuate edge and whose apex-opposing side forms a superior elevated generally arcuate rim, the body composed of flexible and resilient material; an elongate member, composed of flexible and resilient material, extending from each of the first and the second end of the body and terminating in a free end; the elongate member incorporating at least one flexible and resilient co-axial anchoring protuberance positioned along the length. The resilient co-axial protuberances may be generally conical in shape with their respective apexes pointing away from the body. The prosthetic meniscus implant may further comprise reinforcing fibers incorporated into the body and the elongate members.

Embodiments also include a method for implanting a prosthetic meniscus implant comprising obtaining a prosthetic meniscus implant manufactured from resilient polymeric material comprising a generally semi-circular toroidal body having a first end and a second end and having a generally wedge shaped cross section whose apex generates an inferior generally arcuate edge and whose apex-opposing side forms a superior elevated generally arcuate rim, the body composed of flexible and resilient material; an elongate member, composed of flexible and resilient material, extending from each of the first and the second end of the body and terminating in a free end; the elongate member incorporating at least one flexible and resilient co-axial anchoring protuberance positioned along the length, drilling a plurality of anchoring through-holes having a proximal open end and a distall open end in the tibia configured and positioned to allow the protuberances of the prosthetic meniscus implant to pass through holes when the elongate members are pulled through the proximal end when compressed but not allow re-passage of the protuberance once it exits the distal open end; inserting each of the elongate members through its respective the anchoring through-hole and successively compressing one or more the resilient co-axial protuberances as they pass through the respective the anchoring through-hole until the resilient co-axial protuberance exits the distall end of respective the anchoring through-hole thereby permitting the protuberance to reassume its uncompressed shape; wherein the the prosthetic meniscus implant is anchored by the the protuberances retained by the the anchoring through-holes. The method further may include anchoring through-holes which are a constant diameter for their respective entire depth. Alternatively, the method may include the employment of anchoring through-holes comprise a first and a second co-axial portion, the first portion, having a first diameter, and a depth extending from proximal opening for a depth less than the depth to the distal opening, the second portion, having a second diameter, and a depth extending from the maximum depth of the the first portion to the depth of distal opening; the first diameter greater than diameter of the elongate member and less than diameter of uncompressed the protuberance; the second diameter greater than diameter of uncompressed the protuberance.

In a further embodiment a method for selling a prosthetic meniscus implant manufactured from resilient polymeric material comprising a generally semicircular toroidal body having a first end and a second end and having a generally wedge shaped cross section whose apex generates an inferior generally arcuate edge and whose apex-opposing side forms a superior elevated generally arcuate rim, the body composed of flexible and resilient material; an elongate member, composed of flexible and resilient material, extending from each of the first and the second end of the body and terminating in a free end; the elongate member incorporating at least one flexible and resilient co-axial anchoring protuberance positioned along the length; and comprising the steps of informing a medical practitioner of the benefits and availability of the prosthetic meniscus implant; instructing the the medical practitioner the method for implanting the prosthetic meniscus implant; providing the prosthetic meniscus implant.

### BRIEF DESCRIPTION OF DRAWINGS

FIG. 1 illustrates a slightly elevated side view of prosthetic meniscus device according to one embodiment of the present invention;

FIG. 2 illustrates a perspective view of the prosthetic meniscus device as shown in FIG. 1 from a slightly higher elevation;

FIG. 3 illustrates a slightly elevated side view of another prosthetic meniscus device according to another embodiment of the present invention;

FIG. 4A illustrates a top exposed view of the tibial plateau prepared with surgically drilled bore holes;

FIG. 4B illustrates a top exposed view of the tibial plateau prepared with surgically drilled bore holes having multiple sized diameter sections;

FIG. 4C illustrates a top exposed view of the tibial plateau with the prosthetic meniscus device according to one embodiment of the present invention implanted into the surgically drilled bore holes of FIG. 4B.

FIG. 5A illustrates a perspective view of the tibia showing surgically drilled bores according to FIG. 4B with entrance holes on the plateau; and

FIG. 5A illustrates the perspective view of the tibia shown in FIG. 5A having a prosthetic meniscus device according to one embodiment of the present invention implanted into the surgically drilled bore holes; and

FIG. 6 illustrates an exploded view of the interior of a typical tibia as shown in either FIG. 4C or FIG. 5B showing the interface between a larger diameter of the bore hole and the smaller diameter of the bore hole with an anchor lead according to one embodiment of the present invention implanted therein.

### DETAILED DESCRIPTION OF THE INVENTION

Embodiments provide an integrated prosthetic meniscus device and anchoring system that mimics the mechanical characteristics of natural meniscus. The anchoring system according to the present embodiment locks the prosthetic meniscus device directly to the tibia to mimic the securing ligaments of natural meniscus. The prosthetic meniscus is fitted with elongated anchoring leads having locking anchors designed to engage and lock into a surgically drilled anchor hole in the tibia. The prosthetic device preferably includes an outer surface made from a lubricious material, again to mimic that characteristics of natural meniscus. The prosthetic meniscus also preferably includes reinforcing fibers to provide durability and further mimic the characteristics of natural meniscus under stresses and loads typical of a human knee joint.

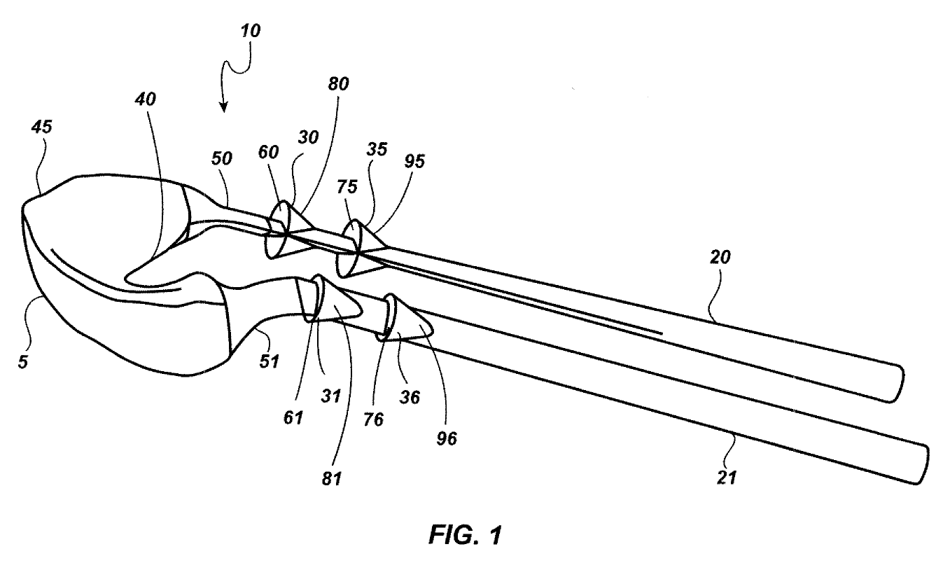

Referring now to FIG. 1, a prosthetic meniscus device 10 having a body portion 5 and two elongated anchoring leads 20, 21 is illustrated. Body portion 5 preferably mimics the wedge shape of natural meniscus and includes a wider rim section 45 and a narrower apex section 40. Each elongated anchoring lead 20, 21 is integrated with body portion 5 at connection points 50 and 51 respectively, preferably in such a manner to mimic the mechanical characteristics of natural mencisii and ligament integration and physiology in the knee joint.

Elongated anchoring leads 20, 21 further include at least one set of anchoring protuberances 30 and 31. In some embodiments, as shown in FIG. 1, elongated anchoring leads include a second set of anchoring protuberances 35 and 36 respectively. Anchor leads 20, 21 may also include three or more anchor protuberances as discussed in more detail below.

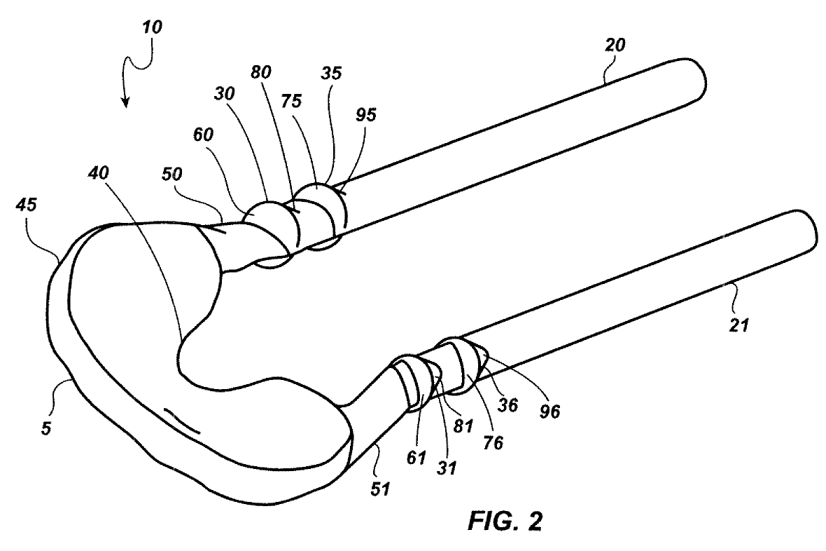

Anchoring protuberances 30, 31, 35 and 36 each include a flat anchoring surface 60, 61, 75 and 76, respectively, (as shown in FIG. 2) where each surface 60, 61, 75 and 76 is positioned substantially perpendicular to the central axis of each elongated anchoring lead 20, 21. Each anchoring protuberance 30, 31, 35 and 36 also includes conical surfaces 80, 81, 95 and 96 respectively, which conical surfaces 80, 81, 95 and 96 slope away from portion 5, toward the distal end of anchor leads 20, 21.

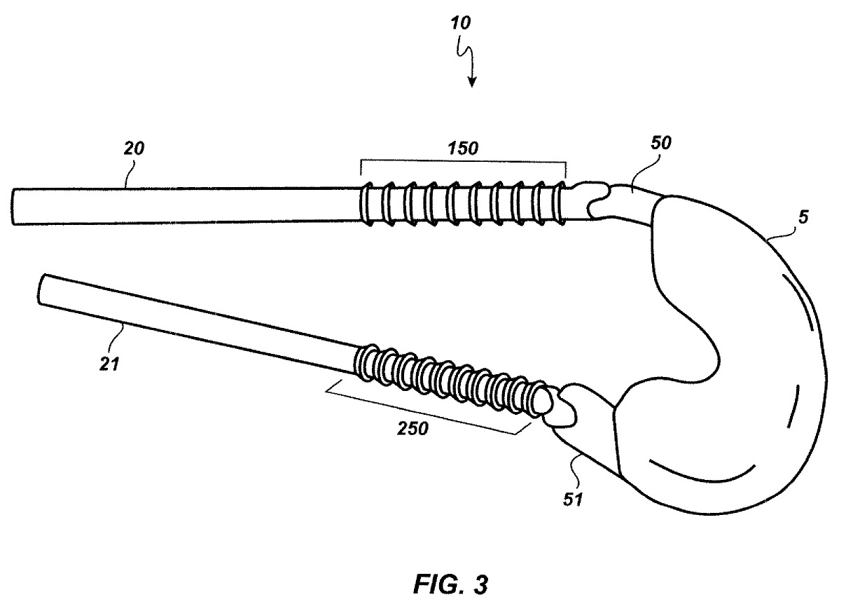

FIG. 3 illustrates an alternative embodiment of the present invention showing anchoring leads 20, 21 having threads 150, 250. Threads 150, 250 begin substantially close in proximity to integration points 50 and 51 respectively and may continue substantially to the end of each anchoring lead 20, 21. As will be shown below anchoring leads 20, 21 are thread through surgically drilled bore holes in the tibia. Threads 150, 250 are sized and shaped to frictionally engage the interior of the bore holes such that prosthetic meniscus device 10 is substantially secure in place on the tibia plateau.

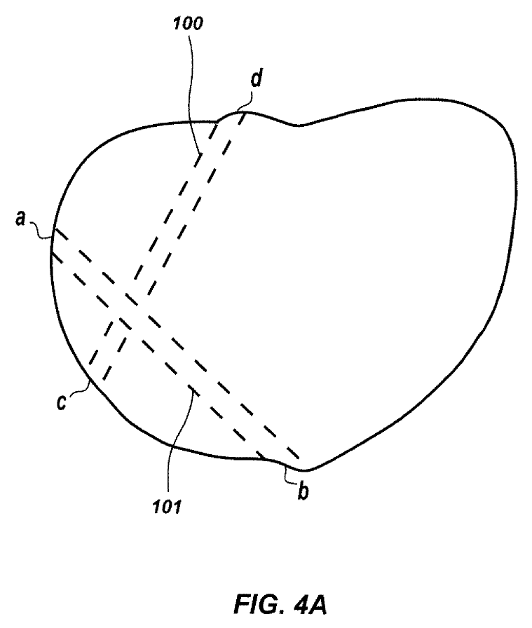

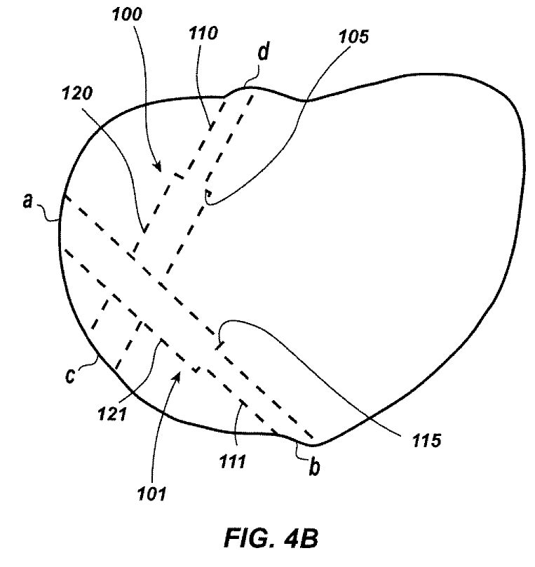

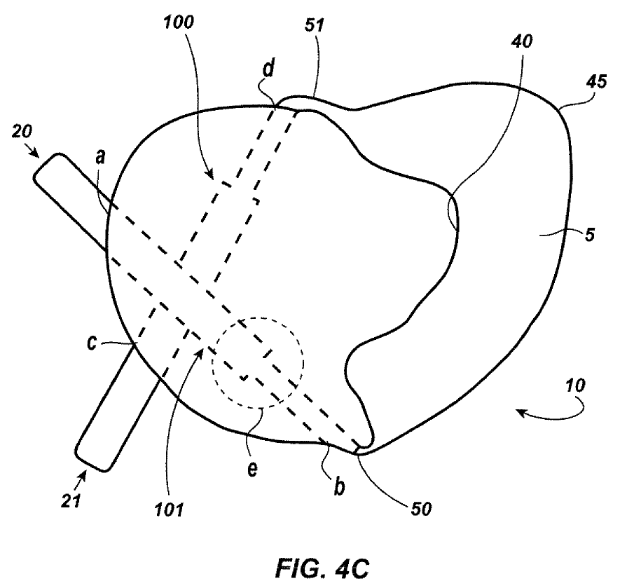

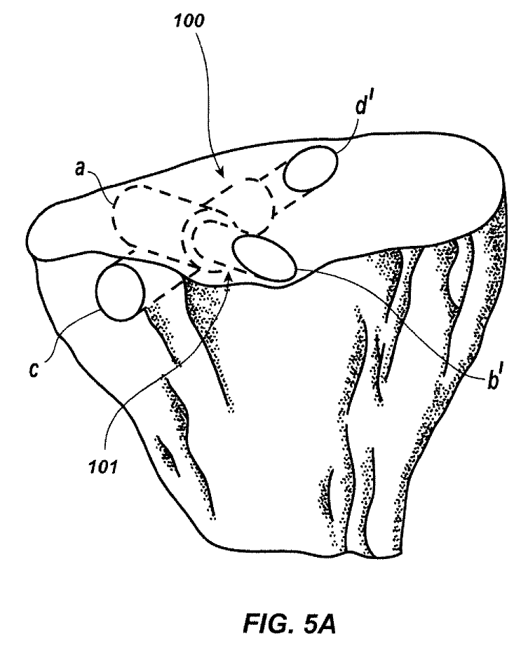

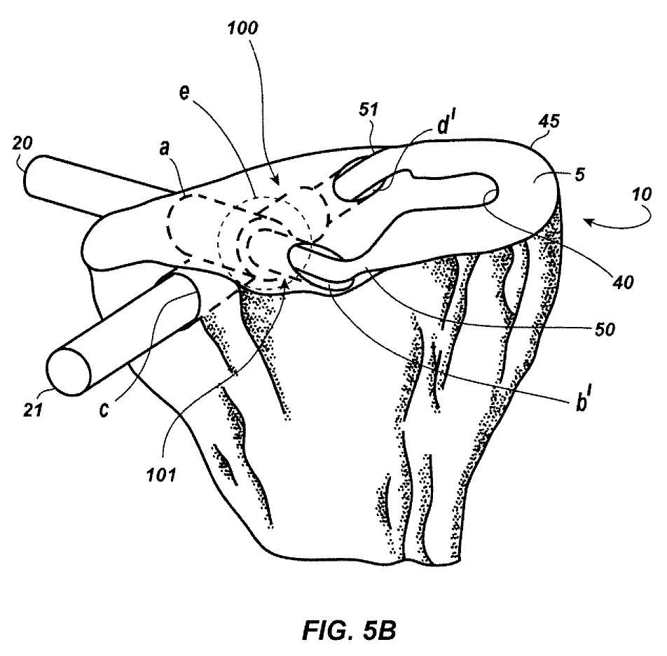

FIGS. 4A and 4B show a top exposed view of the tibial where surgically drilled bore holes 100, 101 have been drilled shown by hatched lines. The angle, depth and width of the bore holes are selectively determined by the surgeon as part of an overall surgery strategy that may be customized to fit the patient along with the size and shape of the prosthetic meniscus device to be implanted. In FIG. 4A, bore holes 100, 101 are shown as cross bore holes each extending from one side wall of tibia, (a, c) across and to another side of the tibia (b, d). In some embodiments, as shown in FIGS. 5A and 5B, bore holes 100, 101 may start at the side wall of the tibia (a, b) and they may end on the tibia plateau (b′, d′).

FIG. 4B shows bore holes 100, 101 where the bore holes are drilled having different diameters along their respective lengths. Bore sections 110 and 111 each have a smaller diameter than bore sections 120 and 121. In this embodiment it is preferable that bore holes 100, 101 have anchoring surfaces 105 and 115 respectively positioned at the interface of bore sections 110 and 120 and 111 and 121. Anchoring surfaces 105, 115 preferably are flat surfaces drilled into the tibia positioned perpendicular to the axis of the bore holes 100, 101. In some embodiments, anchoring positions 105 and 115 may be positioned near or at the surface of the tibia wall (a, c).

FIG. 4C shows the tibia as shown in FIG, 4B with the prosthetic meniscus device implanted therein. Anchor lead 20 has been thread through bore hole a-b such that integration point 50 is snugly adjoining the tibia just outside of bore hole entrance b. Likewise, anchor lead 21 has been thread through bore hole c-d such that integration point 51 is snugly adjoining bore hole entrance d. Each anchor lead is pulled so that prosthetic meniscus device 5 is firmly seated on the tibia plateau with apex 40 directed to the interior of the tibia and rim 45 tracking the circumference of the tibia such then when the femoral condyle is repositioned in its natural location the prosthetic meniscus device will support and move with both the tibia and the femoral condyle in a natural manner.

FIG. 5A shows a perspective view of the tibia bore boles 100, 101 drilled in a slightly different location. Entrance holes c and d instead of being on the wall of the tibia may be drilled into the substantially horizontal tibia plateau. It will be understood that the location of the bore holes 100 and 101, their respective entrances a and b, and their respective exits, c and d, is entirely up to the discretion of the surgeon directing the implantation of prosthetic meniscus device 10. The location of each will be the result of a surgical plan taking into account many factors including bone density, age and weight of the patient as well the general health of the knee joint.

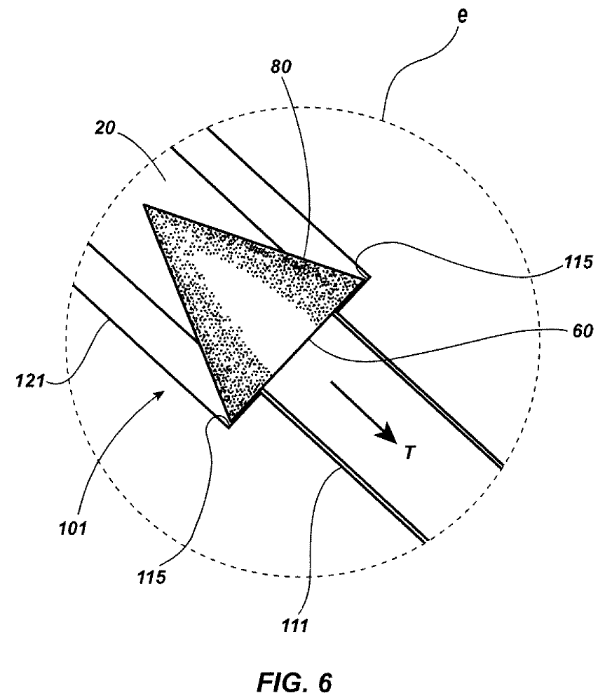

FIG. 5B shows the tibia as described in FIG. 5A with prosthetic meniscus device implanted therein. FIG. 6 shows an exploded view of area ‘e’ as shown in FIGS. 4C and 5B. Anchor leads 20, 21 are preferably made from pliable resilient material such that when anchor leads 20, 21 are put under tensile stress, they will elongate. As shown in FIG. 6, anchor lead has been pulled through bore hole 101 such that anchor protuberance 30 is seated just past the interface between larger diameter bore 121 and smaller diameter bore 111, namely at anchor surface 115. Once anchor protuberance 20 has been pulled past anchoring surface 115, flat anchoring surface 60 abuts anchoring surface 115 and locks anchor lead 20 into place under tension ‘T’. Since meniscus device is made from a resilient material, anchor leads 20, 21 will tend to reform into their original shape and length. As such, tension ‘T’ acts to draw anchor lead 20 back through the bore hole 101 and locks anchor 30 into place against anchoring surface 115. In this view, using device 10 as shown in FIG. 1, anchor lead 20 has been pulled through bore hole 101 such that anchor protuberance 30 is seated at interface section 115 and anchor protuberance 35 (not shown) has also been pulled through bore hole 101. Anchor protuberance 35 (not shown) is positioned upstream of interface 115 in larger diameter bore 121.

As discussed above, FIG. 1 shows anchor leads 20, 21 having two anchor protuberances each, 30, 35 and 31, 36 respectively. However it is entirely possible that in other embodiments anchor leads 20, 21 include three, four or more anchor protuberances in order to fit a particular surgery plan. Furthermore, while the present embodiment is described with respect to bore holes 100 and 101 having only two sections with different diameter, it is entirely possible and within the purview of the present invention that bore holes 100, 101, both or either, include three or more different sections each have a different diameter in order to create a stepped locking system. In this case, it is also entirely within the scope of this disclosure that the anchor protuberances may have corresponding and varying diameters according to the intended bore hole diameter within which each may be respectively seated.

While not shown, when anchor leads 20, 21 are being pulled through bore holes 100, 101 respectively, all anchor protuberances are deformed to fit into the smaller diameter bores 110 and 111 respectively. Each bore hole 110, 111 is drilled with a large enough diameter to ensure that each anchor lead 20, 21 passes through relatively friction free, while each anchor protuberance engages the wall of each bore with a minimum amount of friction. While being pulled through bore holes 110, 111, the anchor protuberances will undergo deformation and once past interface 105, 115 and position into bore holes 120, 121, the anchoring protuberances reform into their original shape such anchoring surfaces 60, 61, 75 and 76 will engage interface surface 105, 115 and lock anchoring leads 20, 21 into place inside the tibia.

In embodiments where there are a series of anchor protuberances, as each anchor protuberance pass the interface sections 105 and 115 they are permanently engaged past this position. If the surgeon desires a tighter fit of the device 10, anchor leads 20, 21 may he pulled through such additional anchor protuberances are pulled past interface sections 105 and 115 hence drawing anchor leads 20, 21, and device 10, into a tighter position with respect to the tibia.

In an alternative embodiment, such as shown in FIG. 3, anchor leads 20, 21 have threads 150 and 250 respectively. Threads 150, 250 maybe pulled though bore holes 100, 101 as shown in FIG. 4A where bore holes 100, 101 have a constant diameter. Threads 150, 210 preferably have a diameter such that they slide into bore holes 100, 101 with a minimum amount of friction but once seated in bore holes 100, 101 they frictionally en-age the walls of bore holes 100, 101 such that they cannot be removed.

### Statement Regarding Preferred Embodiments

While the invention has been described with respect to preferred embodiments, those skilled in the art will readily appreciate that various changes and/or modifications can be made to the invention without departing from the spirit or scope of the invention as defined by the appended claims. All documents cited herein are incorporated by reference herein where appropriate for teachings of additional or alternative details features and/or technical background.

What is claimed is:

1. A prosthetic meniscus implant manufactured from resilient polymeric material comprising:
a generally semi-circular toroidal body having a first end and a second end and having a generally wedge shaped cross section whose apex generates an inferior generally arcuate edge and whose apex-opposing side forms a superior elevated generally arcuate rim, said body composed of flexible and resilient material;
an elongate member, composed of flexible and resilient material, extending from each of said first and said second end of said body and terminating in a free end;
said elongate member incorporating at least one flexible and resilient co-axial anchoring protuberance positioned along the length.

2. A prosthetic meniscus implant of claim 1 where the said resilient co-axial protuberances are generally conical in shape with their respective apexes pointing away from the said body.

3. A prosthetic meniscus implant of claim 1 further comprising reinforcing fibers incorporated into said body and said elongate members.

4. A method for implanting a prosthetic meniscus implant comprising:
obtaining a prosthetic meniscus implant manufactured from resilient polymeric material comprising:
a generally semi-circular toroidal body having a first end and a second end and having a generally wedge shaped cross section whose apex generates an inferior generally arcuate edge and whose apex-opposing side forms a superior elevated generally arcuate rim said body composed of flexible and resilient material; an elongate member, composed of flexible and resilient material, extending from each of said first and said second end of said body and terminating in a free end; said elongate member incorporating at least one flexible and resilient co-axial anchoring protuberance positioned along the length;
drilling a plurality of anchoring through-holes having a proximal open end and a distall open end in the tibia configured and positioned to allow said protuberances of said prosthetic meniscus implant to pass through holes when said elongate members are pulled through the proximal end when compressed but not allow re-passage of the protuberance once it exits said distal open end;
inserting each of said elongate members through its respective said anchoring through-hole and successively compressing one or more the resilient co-axial protuberances as they pass through the respective said anchoring through-hole until said resilient co-axial protuberance exits the distall end of respective said anchoring through-hole thereby permitting said protuberance to reassume its uncompressed shape;
wherein the said prosthetic meniscus implant is anchored by the said protuberances retained by the said anchoring through-holes.

5. The method of claim 4 where each of the said anchoring through-holes are constant diameter for their respective entire depth.

6. The method of claim 4 where each of the said anchoring through-holes comprise a first and a second co-axial portion, said first portion, having a first diameter, and a depth extending from proximal opening for a depth less than the depth to the distal opening, said second portion, having a second diameter, and a depth extending from the maximum depth of the said first portion to said depth of distal opening;
said first diameter greater than diameter of said elongate member and less than diameter of uncompressed said protuberance;
said second diameter greater than diameter of uncompressed said protuberance.

7. A method for selling a prosthetic meniscus implant manufactured from resilient polymeric material comprising:
a generally semi-circular toroidal body having a first end and a second end and having a generally wedge shaped cross section whose apex generates an inferior generally arcuate edge and whose apex-opposing side forms a superior elevated generally arcuate rim, said body composed of flexible and resilient material; an elongate member, composed of flexible and resilient material, extending from each of said first and said second end of said body and terminating in a free end; said elongate member incorporating at least one flexible and resilient co-axial anchoring protuberance positioned along the length;
comprising the steps of:
informing a medical practitioner of the benefits and availability of said prosthetic meniscus implant;
instructing the said medical practitioner the method for implanting said prosthetic meniscus implant; providing said prosthetic meniscus implant.
